(19) United States
(12) Patent Application Publication
(10) Pub. No.: US 2025/0059561 A1
(43) Pub. Date: Feb. 20, 2025

(54) Method for screening a signal peptide for efficient expression and secretion of a heterologous polypeptide in mammalian cells

(71) Applicant: Helsingin yliopisto, Helsingin yliopisto (FI)

(72) Inventors: Ville Paavilainen, Helsingin yliopisto (FI); Juho Kellosalo, Helsingin yliopisto (FI); Paul Carlson, Helsingin yliopisto (FI); Katja Rosti, Helsingin yliopisto (FI); Maryna Green, Helsingin yliopisto (FI); Rahul Nanekar, Helsingin yliopisto (FI)

(21) Appl. No.: 18/721,961

(22) PCT Filed: Dec. 23, 2022
(86) PCT No.: PCT/FI2022/050871

§ 371 Date: Jun. 20, 2024

(30) Foreign Application Priority Data
Dec. 23, 2021 (FI) 20216346

(51) Int. Cl. C12N 15/86; C12N 15/10

(52) U.S. Cl. CPC C12N 15/86; C12N 15/1093; C12N 2740/15043; C12N 2830/003

(57) ABSTRACT

The present disclosure is directed to a method for screening a signal peptide for efficient expression and secretion of a heterologous polypeptide in mammalian cells, the method comprising the steps of: providing a pool of viral expression vectors encoding a polypeptide of interest with various candidate signal peptides, wherein each viral expression vector of said pool comprises at least a polynucleotide encoding a fusion protein, said polynucleotide comprising: i) a promoter, ii) a sequence encoding a signal peptide, iii) a sequence encoding the polypeptide of interest, iv) optionally a sequence encoding an epitope tag, and v) a sequence encoding a C-terminal signal peptide for glycosylphosphatidylinositol, GPI, attachment or a sequence encoding a transmembrane domain; transforming host cells with said pool of viral vectors so that each host cell is preferably transformed on average by only one viral vector from said pool; expressing said fusion protein in said host cells in order to produce fusion proteins which are GPI-anchored to the host cell surface or alternatively which are anchored to the host cell surface by a transmembrane domain of said fusion protein; contacting said host cells with a first binding reagent, preferably an antibody, specifically binding to said polypeptide of interest or alternatively if said epitope tag is present in the fusion protein with a first binding reagent specifically binding to said epitope tag, wherein said binding reagent is optionally labelled with a fluorescent label or other means of detection; dividing the transformed host cells into at least two groups based on the fluorescence characteristics of each host cell; and performing next generation sequencing to the group of host cells showing the most efficient fusion protein expression in order to identify an optimal signal peptide for the polypeptide of interest in the host cell. The present disclosure is also directed to a vector, a host cell or a DNA library for use in said method.

### FIELD

This disclosure relates to the field of recombinant protein production. In particular, the present disclosure relates to a method for selecting a specific signal peptide that efficiently secretes a protein of interest out of host cells from a variety of signal peptides.

### BACKGROUND

Protein translocation is the dynamic mechanism underlying the shipment of about 30% of all cellular proteins across and into the plasma membrane (Gemmer, Forster 2020). This process is facilitated by a translocon, a multi-subunit protein complex located on endoplasmic reticulum (ER) membrane. Universally conserved heterotrimeric protein channel Sec61 forms the core of the translocon.

When the ribosomes start secretory protein synthesis in the cytosol of eukaryotic cells, an ER signal peptide (SP) sequence located at the amino terminus of a nascent polypeptide chain directs the ribosome to the ER membrane. The SP sequence of the nascent protein is recognized by the signal recognition particle (SRP), and the growing polypeptide is translocated across the ER membrane. Thus, SPs function as zip codes marking the protein secretion pathway and the protein target location (Blobel, Dobberstein 1975) Based on computational and experimental studies the SP sequences are divided into three characteristic regions: positively charged amino acid containing hydrophilic N-terminal region, a hydrophobic core region, and a C-terminal region with a cleavage site for a signal peptidase that usually contains polar amino acid residues (von Heijne 1985).

The signal peptide-mediated translocation of secretory proteins into the lumen of the ER has been identified as a bottleneck within the secretory pathway and thus represents a key issue that needs to be resolved to achieve robust production of recombinant proteins. It has been shown that signal peptides are extremely heterogeneous, and many signal peptides are functionally interchangeable even between different species (Tan, Ho et al. 2002). On the other hand, different signal peptides can exert profoundly different effects on protein secretion and function of the produced proteins (Kober et al. 2013). Thus, the efficiency of protein secretion can be strongly affected by the signal peptide sequence These observations are highly indicative of the importance of signal peptide optimization when aiming to produce maximal amounts of recombinant proteins in a mammalian system.

The current signal peptide-optimization methods rely on laborious testing of individual signal peptides (Kober et al. 2013), which either limits the number of sequences-that can be tested, and thus makes discovery of optimal signal peptides unlikely, or takes prohibitively long time.

### SUMMARY

In this patent application, we present a screening platform that allows rapid and efficient high-throughput screening of thousands of signal peptides for finding optimal ones that allow high-level production of therapeutically relevant proteins in mammalian cells (FIGS. 1 and 6). The present disclosure contains detailed use of the platform for the signal-peptide optimization of two model proteins: super-folder GFP and Coagulation Factor VII (FVII), a therapeutic protein which is utilized for treating hemophilia-like bleeding disorder caused by factor VII or IX deficiency.

In addition to the physical signal peptide screening platform, we have also designed artificial intelligence methods for predicting signal peptides that are optimal for the production of different biotechnologically important proteins. This appears to be an area of interest in the present field of technology so this approach will likely form an important part of the present disclosure.

In an aspect of the present disclosure, we provide a method for screening signal peptides for efficient expression and secretion of a heterologous polypeptide in mammalian cells, the method comprising the steps of:
a) providing a pool of viral expression vectors encoding a polypeptide of interest with various candidate signal peptides, wherein each viral expression vector of said pool comprises at least a polynucleotide encoding a fusion protein, said polynucleotide comprising: i) a promoter, ii) a sequence encoding a signal peptide, iii) a sequence encoding the polypeptide of interest, iv) optionally a sequence encoding an epitope tag, and v) a sequence encoding a C-terminal signal peptide for glycosylphosphatidylinositol, GPI, attachment or a sequence encoding a transmembrane domain;
b) transforming host cells with said pool of viral vectors so that each host cell is preferably transformed on average by only one viral vector from said pool;
c) expressing said fusion protein in said host cells in order to produce fusion proteins which are GPI-anchored to the host cell surface or alternatively which are anchored to the host cell surface by a transmembrane domain of said fusion protein;
d) contacting said host cells with a first binding reagent, preferably an antibody, specifically binding to said polypeptide of interest or alternatively if said epitope tag is present in the fusion protein with a first binding reagent specifically binding to said epitope tag, wherein said binding reagent is optionally labelled with a fluorescent label or other means of detection;
e) optionally contacting said host cells with a second binding reagent, preferably an antibody, labelled with a fluorescent label if said first binding reagent is not a labelled binding reagent, wherein said second binding reagent is specifically binding to said first binding reagent;
f) dividing the transformed host cells into at least two groups based on the fluorescence characteristics of each host cell; and
g) performing next generation sequencing to the group of host cells showing the most efficient fusion protein expression in order to identify an optimal signal peptide for the polypeptide of interest in the host cell.

In another aspect of the present disclosure, we provide a viral expression vector comprising at least a polynucleotide encoding a fusion protein, said polynucleotide comprising: i) a promoter, ii) a sequence encoding a signal peptide, iii) a sequence encoding a polypeptide of interest, iv) optionally a sequence encoding an epitope tag, and v) a sequence encoding a C-terminal signal peptide for glycosylphosphatidylinositol, GPI, attachment.

In further aspects of the present disclosure, we provide 1) a host cell comprising a viral vector according to the present disclosure, and 2) a DNA library comprising multiple viral vectors according to the present disclosure, wherein the vectors encode various candidate signal peptides for a polypeptide of interest.

### BRIEF DESCRIPTION OF THE DRAWINGS

FIG. 1. A graphical representation of the present signal peptide (SP) screening platform. A) The schematic of the fate of GPI-anchored, epitope-tagged protein of interest (POI-EPI-GPI) with a functional and a non-functional signal peptide. Functional signal peptide targets the protein of interest to the ER where the protein undergoes folding and maturation. Correctly folded and processed POI-EPI-GPI gets trafficked to the cytoplasmic surface, on which the protein can be labelled with an epitope tag or folded POI-binding antibody. If the signal peptide does not facilitate the POI's ER-targeting or its maturation or folding (i.e. it is not functional), the POI will either stay (faulty targeting) or be directed into the cytoplasm (faulty folding/maturation) by ER-associated degradation pathway and be degraded by cytoplasmic proteasomes. B) The workflow of the signal peptide screening process. Signal peptide encoding DNA oligo pools will be synthesized and preferably cloned in batch into the POI-EPI-GPI-encoding lentiviral transfer plasmids. Human or Chinese hamster ovary (CHO) cells will be transduced with the SP-POI-EPI-GPI encoding constructs with a low M.O.I. so that, on average, one transduced cell will only contain one SP-POI-EPI-GPI encoding construct. The transduced cells, which preferably express, besides of the SP-POI-EPI-GPI, an iRFP protein as a transduction marker, will be stained with an epitope tag-recognizing antibody (Anti-EPI), and then sorted on the basis of the Anti-EPI signal. Alternatively, a folded protein-recognizing antibody can also be used for cell staining. SP insert coding regions of the genome integrated lentiviral constructs will be amplified and then identified with next generation sequencing. Comparison of sequencing reads of SPs from cell populations which show strong and weak Anti-EPI/Anti-folded protein signal allows identification of SPs which facilitated the highest level of POI production. As an enrichment control prior to the next generation sequencing, the specific enrichment of signal peptides in sorted pools will be assessed with qPCR.

FIG. 2. (A) Lentiviral expression cassette. Variable regions 1 and 2 where signal peptide (SP) library and protein of interest (POI) are cloned. (B) Schematic representation of a translated protein polypeptide that is secreted outside the expression host and displayed onto the plasma membrane via GPI/TM achor. LTR: long terminal repeats, Promoter: inducible promoter, SP: signal peptide, POI: Protein of interest, GPI: glycophosphatidylinositol, TM: trans-membrane domain, IRES: internal ribosome entry site, Transduction marker: fluorescent protein, N: Amino terminus, C: Carboxy terminus. (C) In the present experiments, the construct comprises Coagulation Factor VII (FVII) as POI and the epitope tag is 3xFLAG-tag.

FIG. 3. Identification of enriched SPs with qPCR/RT-PCR. In step 1, GFP expressing CHO cells are sorted into two separate tubes based on their GFP expression levels; Hi-GFP (Q2) and Low-GFP (Q3). In step 2 and 3, Genomic DNA from the sorted cells is extracted and used as a template in qPCR/RTPCR. Site-specific primers are used to amplify the signal peptide region by qPCR. Low-GFP cells contained azurocidin preprotein SP. GFP: green fluorescent protein, RFP: red fluorescent protein, gDNA: genomic DNA, Hi-GFP: higher level of GFP expressing cells, Low-GFP: low level of GFP expressing cells, qPCR: quantitative polymerase chain reaction, RT-PCR: reverse transcription PCR.

FIG. 4. Specific SPs influence Coagulation Factor VII (FVII) expression levels. (A) Western-blot of Anti FLAG anti-body stained HEK293T cells transduced with construct 1: ORI SP FVII and construct 2: IgK SP FVII (expected size of the protein is 50 kDa). (B) Quantification of the relative expression levels based on the 50 kDa bands' average intensities.

FIG. 5. Anti-body staining of GPI-anchored FVII allows sorting of differentially stained cells. (A) FACS plot of negative control: HEK293T cells induced with doxycycline and stained with both: primary (anti-FLAG) and secondary (AlexaFluor 647) antibodies. (B) FACS plot of negative control: SP Library FVII transduced HEK293T cells induced with doxycycline and stained with the secondary (AlexaFluor 647) anti-body only. (C) FACS plot of SP Library FVII transduced HEK293T cells induced with doxycycline and stained with both: primary (anti-FLAG) and secondary (AlexaFluor 647) anti-bodies, 1.2% of cells give both transduction marker (tRFP) and secondary antibody signals (highlighted with the dotted lined box within Q2).

FIG. 6 shows the step-by-step workflow used in the signal peptide screening platform of the present disclosure.

### EMBODIMENTS

The term “signal peptide” means herein a peptide that is a part of the N-terminus of a secretory protein that is secreted outside a cell and thus passes through the cell membrane. The signal peptide is usually composed of approximately 10 to 30 amino acids, and is subsequently cleaved and removed by a protease specific for the cell membrane, and only the secretory protein is transferred outside the cell. Signal peptides serve as targeting signals, enabling cellular transport machinery to direct proteins to specific intracellular or extracellular locations. To date, more than 4000 signal peptides present in eukaryotic cells are known. DNA libraries encoding signal peptides are disclosed, e.g., in WO2021045541Al and KR20210028116A.

The term “protein of interest” or “POI” in the present specification means a protein that is intended to be produced with high efficiency by using a suitable host cell. The POI is preferably a therapeutically or diagnostically significant protein such as an antibody.

The term “epitope tag” or EP refers herein to a technique in which a tag (typically 6 to 30 amino acids) is fused to a recombinant protein by placing sequence encoding the epitope within the same open reading frame of the protein by means of genetic engineering. By choosing an epitope tag for which an antibody is available, the technique makes it possible to detect tagged proteins for which otherwise no antibody is available. By selection of the appropriate epitope tag and antibody pair, it is possible to find a combination with properties that are suitable for the desired experimental application, such as Western blot analysis, immunoprecipitation, immunochemistry, affinity purification, and others. Preferred epitope tags can be selected for example from a group consisting of Histidine tag (His-tag), myc-tag, FLAG-tag, small ubiquitin-like modifier tag (SUMO-tag), a heavy chain of protein C tag (HPC-tag), a calmodulin binding peptide tag (CBP-tag), and a hemagglutinin-tag (HA-tag).

The term “GPI-anchored” refers herein to glycosylphosphatidylinositol (GPI) anchored proteins which are found on the external surfaces of eukaryotic cells These secreted proteins are anchored to the plasma membrane with a GPI moiety covalently attached to the C-terminus of the protein. The GPI moiety consists of the conserved core glycan, phosphatidylinositol and glycan side chains. The structure of the core glycan is EtNP-6Manα2-Manα6-(EtNP)2Manα4-GlNα6-myoIno-P-lipid (EtNP, ethanolamine phosphate; Man, mannose; GlcN, glucosamine; Ino, inositol). In the present disclosure, a C-terminal signal peptide directs a protein to the GPI attachment, see, e.g., EP3389682.

The term “transmembrane domain” refers herein to a hydrophobic alpha helix structure that transverses the host cell membrane. The transmembrane domain may be directly fused to the C-terminal part of the fusion protein encoded by the present vectors. In certain embodiments, the transmembrane domain is derived from an integral membrane protein (e.g., receptor, cluster of differentiation (CD) molecule, enzyme, transporter, cell adhesion molecule, or the like). In preferred embodiments, the transmembrane domain is derived from Type 1 transmembrane proteins exemplified by human VCAM-1 protein (vascular cell adhesion molecule 1). Type I transmembrane proteins are anchored to the lipid membrane with a stop-transfer anchor sequence and have their N-terminal domains targeted to the extracellular space, when a mature form of the protein is located on the cell membrane. Further examples of transmembrane domains according to the present disclosure include, but are not limited to, Tim1, Tim2and Tim3 transmembrane domains, FcR transmembrane domains, and a CD8a transmembrane domain. Further transmembrane domains for use in the present invention are disclosed in EP3389682.

The term “vector” is used herein to refer to a nucleic acid molecule capable of mediating entry of, e.g., transferring, transporting, etc., another nucleic acid molecule into a cell. The transferred nucleic acid is generally linked to, e.g., inserted into, the vector nucleic acid molecule. A vector may include sequences that direct autonomous replication, or may include sequences sufficient to allow integration into host cell DNA. Useful vectors include, for example, plasmids, cosmids, and viral vectors. Useful viral vectors include, e.g., replication defective retroviruses, adenoviruses, adeno-associated viruses, and lentiviruses. As will be evident to one of ordinary skill in the art, viral vectors may include various viral components in addition to nucleic acid(s) that mediate entry of the transferred nucleic acid. Thus, the term viral vector may refer either to a virus or viral particle capable of transferring a nucleic acid into a cell or to the transferred nucleic acid itself.

In an embodiment, the present disclosure is directed to a method for screening a signal peptide for efficient expression and secretion of a heterologous polypeptide in mammalian cells, the method comprising the steps of:
a) providing a pool of viral expression vectors encoding a polypeptide of interest with various candidate signal peptides, wherein each viral expression vector of said pool comprises at least a polynucleotide encoding a fusion protein, said polynucleotide comprising: i) a promoter, ii) a sequence encoding a signal peptide, iii) a sequence encoding the polypeptide of interest, iv) optionally a sequence encoding an epitope tag, and v) a sequence encoding a C-terminal signal peptide for glycosylphosphatidylinositol, GPI, attachment or a sequence encoding a transmembrane domain;
b) transforming host cells with said pool of viral vectors so that each host cell is preferably transformed on average by only one viral vector from said pool;
c) expressing said fusion protein in said host cells in order to produce fusion proteins which are GPI-anchored to the host cell surface or alternatively which are anchored to the host cell surface by a transmembrane domain of said fusion protein;
d) contacting said host cells with a first binding reagent, preferably an antibody or an aptamer, specifically binding to said polypeptide of interest or alternatively if said epitope tag is present in the fusion protein with a first binding reagent specifically binding to said epitope tag, wherein said binding reagent is optionally labelled with a fluorescent label or other means of detection;
e) optionally contacting said host cells with a second binding reagent, preferably an antibody or an aptamer, labelled with a fluorescent label if said first binding reagent is not a labelled binding reagent, wherein said second binding reagent is specifically binding to said first binding reagent;
f) dividing the transformed host cells into at least two groups based on the fluorescence characteristics of each host cell, preferably by using fluorescence-activated cell sorting. FACS; and
g) performing next generation sequencing to the group of host cells showing the most efficient fusion protein expression in order to identify an optimal signal peptide for the polypeptide of interest in the host cell.

In a preferred embodiment, wherein said pool of viral expression vectors encoding a polypeptide of interest in step a) of the present method is prepared by
synthesizing a library of oligonucleotides encoding various signal peptides and complementary sequences thereof, wherein said oligonucleotides form restriction site overhangs at both free ends of the sequence when bound to a complementary oligonucleotide in the library;
annealing the oligonucleotides in the library with the complementary oligonucleotides present in the library to produce double-stranded DNA fragments with said restriction site overhangs; and
ligating said double-stranded DNA fragments to a viral vector having a suitable cloning site to combine the DNA fragment encoding a signal peptide with a polynucleotide encoding a protein of interest and thus producing a pool of viral expression vectors encoding a polypeptide of interest with various candidate signal peptides.

In a preferred embodiment, wherein the library of oligonucleotides encoding various signal peptides comprises the known signal peptides of eukaryotic, preferably mammalian, bacterial or viral proteins, modifications thereof and/or artificial sequences.

In another preferred embodiment, the method of the present disclosure comprises a further step of
h) cloning a polynucleotide encoding the combination of said optimal signal peptide detected in step g) and the polypeptide of interest to a second expression vector, transforming a host cell with said second vector and producing said polypeptide in said host cell.

In a preferred embodiment, said promoter of the vector is an inducible promoter, preferably a tetracycline controlled promoter.

In another embodiment, the present disclosure is directed to a viral expression vector comprising at least a polynucleotide encoding a fusion protein, said polynucleotide comprising: i) a promoter, ii) a sequence encoding a signal peptide, iii) a sequence encoding a polypeptide of interest, iv) optionally a sequence encoding an epitope tag, and v) a sequence encoding a C-terminal signal peptide for glycosylphosphatidylinositol, GPI, attachment.

In further embodiments, the present disclosure is directed to 1) a host cell comprising a vector according to the present disclosure or 2) a DNA library comprising multiple viral vectors according to the present disclosure, wherein the vectors encode various candidate signal peptides for a polypeptide of interest.

While the following examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the present disclosure. Accordingly, it is not intended that the present invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, that is, a singular form, throughout this document does not exclude a plurality.

Reference throughout this specification to one embodiment or an embodiment means that a particular feature, structure, or characteristic described in connection with the embodiment is included in at least one embodiment of the present disclosure. Thus, appearances of the phrases “in one embodiment”, “in an embodiment” or “in a preferred embodiment” in various places throughout this specification are not necessarily all referring to the same embodiment. Where reference is made to a numerical value using a term such as, for example, about or substantially, the exact numerical value is also disclosed.

### EXPERIMENTAL SECTION

### Construction of pINDUCER Based Mammalian Expression Plasmid

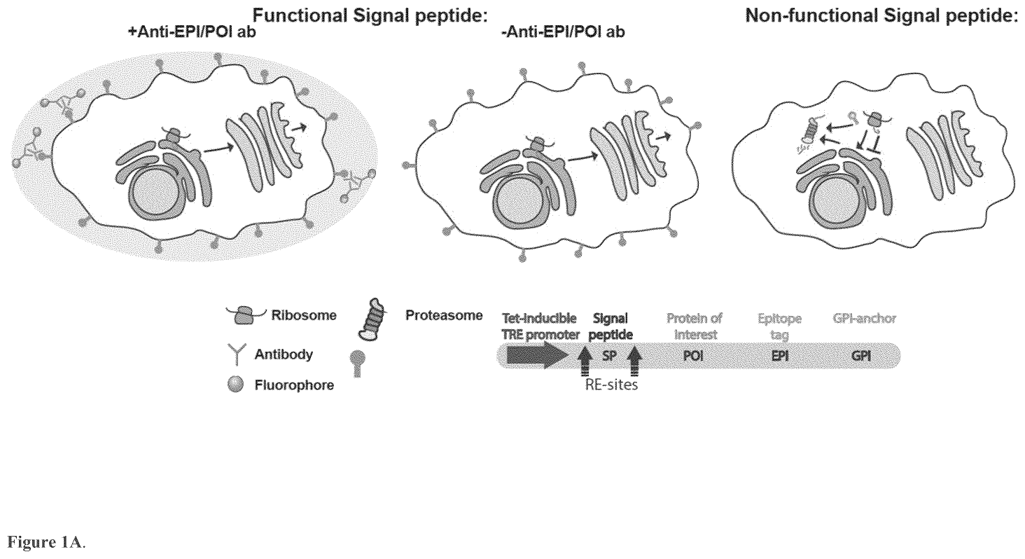

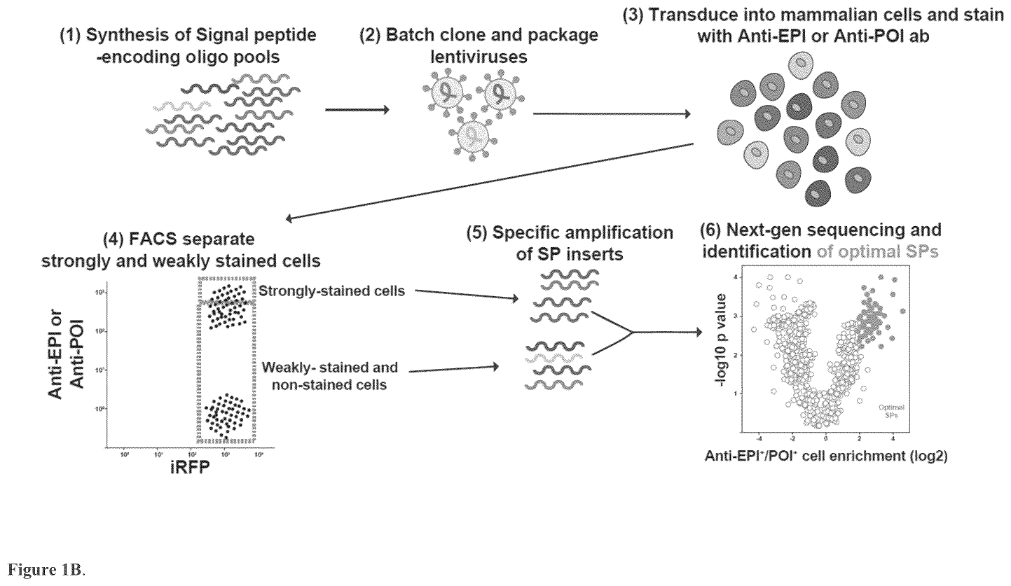

The lentiviral transfer plasmid which allows the expression of the protein-of-interest (POI) in a plasma-membrane anchored form forms the core of our signal peptide-screening platform (FIG. 1). Lentiviral transduction of the expression construct allows us to isolate a single signal peptide-POI combination carrying cell which shows high expression level of POI (FIG. 1). High-throughput screening of signal peptides can be achieved by combining the use of our lentiviral construct with massive scale gene synthesis of signal peptide libraries and use of next generation sequencing for identification of optimal signal peptides from pooled, flow-cytometry sorted cell samples (FIG. 1). This massively parallel signal peptide screening platform allows identification of optimal signal sequences for protein production in a manner that is both faster and more comprehensive than the current signal peptide optimization methods that rely on individual testing of signal peptides.

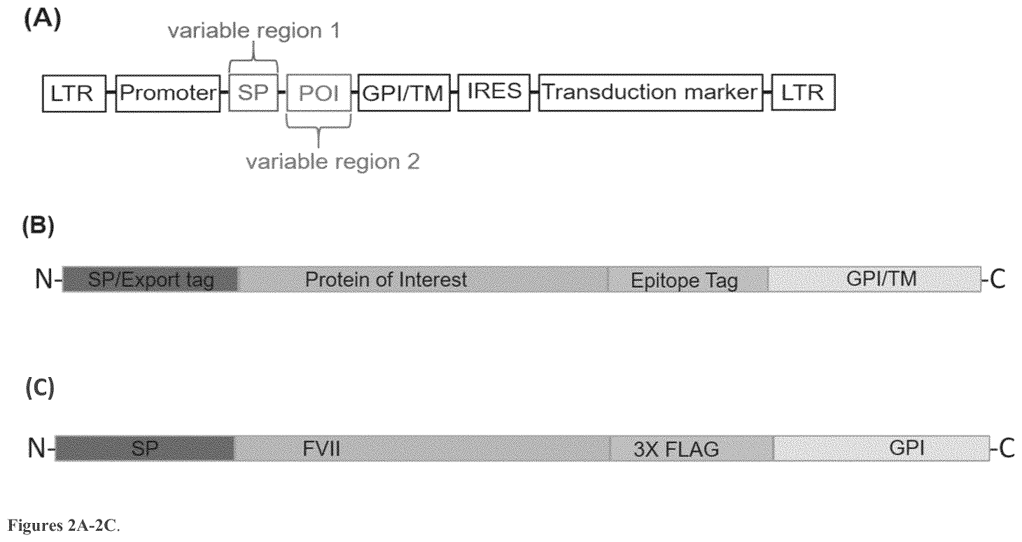

The lentiviral transfer plasmid (FIG. 2) that we used for the genomic integration of the tested signal-peptide-protein-of-interest constructs is based on a pINDUCER11 plasmid (Meerbrey, Hu et al. 2011) in which the original transduction marker (GFP) has been changed to iRFP670 (Shcherbakova, Verkhusha 2013) or tRFP (Strack, Strongin et al. 2008) by amplifying an IRES-tRFP or IRES-iRFP670 insert with overlap extension PCR and then cloning the insert into the vector with AscI and PacI restriction enzyme (NEB) digestion and subsequent ligation with T4 ligase (NEB).

For signal-peptide screening of superfolder-GFP SP-ubiquiting G76V(Ub(G76V))-sfGFP-GPI-anchor construct was assembled with over-lap extension PCR from separate Ub(G76V) (Dantuma, Lindsten et al. 2000), sfGFP (Costantini, Baloban et al. 2015) and GPI-anchor (Rhee, Pirity et al. 2006) inserts. The assembled fusion-protein insert was cloned in place of the tRFP-shRNA insert of the original pINDUCER11 plasmid.

For signal-peptide screening of FVII, DNA constructs containing a hamster codon-optimized, inactive FVII (D302N)-mutant were ordered as SP-3xFLAG-tag-FVII-GPI or SP-HA-tag-FVII-GPI fusion protein gene fragments from Twist Biosciences. These fusion-protein encoding DNA constructs were cloned in place of the tRFP-shRNA insert of the original pINDUCER11 plasmid.

Functional Components of the Modified pINDUCER11 Plasmid:
CMV promoter: Doxycycline inducible promoter driving expression of gene of interest
Enhanced Green Fluorescent Protein (eGFP): Used as a gene of interest in our proof-of-concept experiments.
Internal Ribosome Entry Site (IRES): It enables the translation initiation in cap-independent manner.
Transactivator 3 (rtTA3): Controls the doxycycline induction of the CMV promoter. Under constitutive promoter hUBC.
Human Ubiquitin C promoter (hUBC): Constitutive promoter to drive ectopic iRFP670 or tRFP expression.
iRFP670: Transduction reporter protein under the constitutive promoter hUBC.
tRFP: Transduction reporter protein under the constitutive promoter hUBC
SV40 gene: Plasmid replication region

### Cloning of Signal Peptides Into Expression Plasmid

For directional cloning, SP insert generated by annealing oligonucleotides and circular pINDUCER vector were double-digested with two RE's, MluI and NotI. Additionally, double-digested pINDUCER vector was treated with Shrimp alkaline phosphatase (rSAP) which nonspecifically catalyzed the dephosporylation of 5′ ends to avoid the self-ligation of the vector. However, ligation of DNA fragments require the 5′ phosphate groups to form phosphodiester bonds, double-digested oligonucleotides were subjected to phosphorylation in a thermal cycler using T4 polynucleotide kinase (NEB) in presence of ATP. Thus generated SP insert and linear pINDUCER vector were covalently ligated together using T4DNA ligase (NEB). The entire ligation mixture was transformed into stable3 (Thermo Fisher) chemical competent E. coli cells and grown onto LB agar plates containing ampicillin antibiotic (100 μg/ml). Resulting colonies were screened for the correct SP insert by Sanger sequencing.

### Cell Lines and Their Cultivation Under Standard Conditions

Human embryonic kidney 293 cells (HEK293T) (Thermo-Fisher Scientific) were cultured as adherent monolayers in DMEM containing 10% fetal bovine serum (FBS) and 0.5% L-Glutamine. FreeStyle Chinese hamster ovary (CHO) suspension adapted (CHO-S) cells (Thermo-Fisher Scientific) were grown as a suspension culture in FreeStyle™ CHO media (Thermo-Fisher Scientific). Both the cell lines were cultured under standard conditions at 37° C., 5% CO₂.

### Example 1. Proof-Of-Concept Experiments

### Model Protein 1: Green Fluorescent Protein (GFP)

Following signal peptides were selected for proof-of-concept experiments with sfGFP; SP1 (Interleukin 4), SP2 (Serum Albumin), SP3 fPrP (mut 17-21) SP4 (Azurocidin preprotein) SP5 (Cellulase), SP6 (PrP), SP7 (Vcam), SP8 (FCRL-1).

Aforementioned SPs were cloned into the modified pINDUCER11 (see 2) RE sites for MluI (NEB) and NotI (NEB) were utilized. Detailed cloning strategy is explained here.

For each signal peptide, oligonucleotides containing the respective sequence coding for the signal peptide were synthesized (from Integrated Data Technologies). All the oligonucleotides were flanked by MluI and NotI restriction enzyme sites on 5′ and 3′ ends respectively. Single stranded oligonucleotides were annealed in a thermal cycler (Bio-Rad) to generate a dsDNA insert.

### Model Protein 2: Coagulation Factor FVII (FVII)

Third generation lentiviral transfer plasmid pINDUCER11 containing gene of interest was designed as disclosed above. Two different signal peptides (Table 1), selected on the basis of their differential effects on FVII expression PMID: (Peng, Yu et al. 2016), were cloned into the N-termini of 3xFLAG-tag-FVII-GPI and HA-tag-FVII-GPI fusion constructs.

TABLE 1
Individual natural signal peptides used with FVII

| Signal | | |
|---|---|---|
| peptide | Amino acid sequence | Hamster codon-optimized DNA sequence |
| IgK | METDTLLLWVLLL | ATGGAGACCGACACCCTGCTGCTGTGGGTG |
| | WVPGSTG (SEQ ID | CTGCTGCTGTGGGTGCCCGGCTCCACCGGC |
| | NO: 1) | (SEQ ID NO: 2) |
| ORI | MVSQALRLLCLLL | ATGGTGTCCCAGGCCCTGAGGCTGCTGTGC |
| | GLQGCLA (SEQ ID | CTGCTGCTGGGCCTGCAGGGCTGCCTGGCC |
| | NO: 3) | (SEQ ID NO: 4) |

### a) Production of Lentivirus Expressing Protein of Interest in Mammalian Cells

Third generation lentiviral transfer plasmid pINDUCER11 containing gene of interest was designed as disclosed above. Other lentiviral packaging plasmids pVP157, pVP158, pVP159 and pVP160 were received as a gift from Martin Kampmann/Jonathan Weissmann (Bassik, Kampmann et al. 2013).

The lentivirus production was based on polyethyleneimine (PEI) mediated transfection protocol as described elsewhere (PMID: (Lobato-Pascual, Saether et al. 2013, Bassik, Kampmann et al. 2013). Briefly, cationic polymer PEI containing Transporter 5® Transfection reagent (Polysciences, Germany) was used to transfer and packaging vectors into the HEK293T cells. In total 800-1000μg of transfer plasmid was mixed with packing plasmids. Transfection reagent was diluted in PBS and was mixed with the plasmids. This mixture was incubated at room temperature for 25 min. Dropwise addition of this mixture to adherent HEK293T cell culture assured even distribution. Cells were incubated for three days (72 h) post-transfection. The supernatant containing lentivirus was collected. The supernatant solution was filtered using 0.45μ filtration assembly. The filtered lentivirus was collected in aliquots and stored at 4° C.

### b) Virus Titer Determination in HEK293T and CHO Cells

The protocol for lentivirus titration was adapted from (Tiscornia, Singer et al. 2006).

Day 1: Seed 24-well plates with 500 μl cells (100,000 cells in each well)

Day 3: Four-fold serial dilution of lentiviral stock was prepared in the media.

Virus dilutions: undiluted, 1:4, 1:16, 1:64

The media was removed from the wells of 24-well plate and supplemented with 250 μL of fresh media. Virus dilutions were added (20 μL) in a dropwise manner to the cells, mixed gently and incubated the cells at 37° C. After 2-3 h additional 250 μL of media was added to the wells. Cells were grown for 48 h.

Day 5: The media was discarded from the wells. Cells were washed once with 150 μl of PBS. Cells were dislodged using 30 μl of Trypsin (0.5%) and then mixed with 500 μl fresh media. In another plate 400 μl fresh media was added together with 100 μl cells from day 3. Cells were then incubated at 37° C. for 3 days.

This step was repeated for three additional times. In total, cells were washed and divided for a week. These extensive washing steps were important to remove the virus particles.

Day 14: Cells were prepared for FACS analysis as explained in flow cytometry section below. FACS analysis was performed to determine the percentage of fluorescent reporter (GFP and mRFP) positive cells.

Virus titer (VT=TU/mL, transduction units) was calculated according to the following formula: Transduction Unit (TU/ml)=(N×P)/(V×D)

Where; N=Cell Number in each well used for infection on Day 2; P=percentage of GFP/RFP positive cells (should be 10% ˜20%); V=virus volume used for infection in each well; the V (ml)=20(μl)×10−3 in this protocol; D=dilution fold; TU=transduction unit.

### c) Cell Surface Antibody Staining and Flow-Cytometry

HEK293T and CHOcells were transduced with 3X-FLAG-or HA-tag harboring FVII-fusion protein or sfGFP-fusion protein encoding lentiviruses. Transduced cells were splitted 2 times. 24 h prior to harvesting, cells were induced with 1 μg/ml doxycycline. Cells were harvested with 10 mM EDTA. Cells were pelleted by centrifugation at 2400 rpm for 5 min at room temperature and then resuspended in room-temperature FACS Buffer (1×PBS, 4% FBS, 10 mM EDTA). Sample aliquot was saved for FACS analysis, with the rest pelleted by centrifugation for 2 min at 2400 rpm at room temperature and then resuspended in 1 mL FACS buffer. Centrifugation was repeated and the media was then removed (1 wash). Cell pellet was resuspended in 500 μl primary antibody solution (see Table 2), incubated for 15 min at room temperature and then washed twice with 1 mL FACS buffer. Afterwards, cells were resuspended in 500 μl secondary antibody solution (see Table 2), incubated for 15 min at room temperature, washed twice with 1 mL FACS buffer and finally resuspended in FACS buffer. Just before the FACS measurements, cells were strained twice into FACS tubes and then either analyzed with BD LSRFortessa flow-cytometer or sorted with BD FACSAria II flow-cytometer to low and high Antibody signal showing populations (see Fig.1). The flow-cytometry data was analyzed with FlowJo software (Tree Star, Inc., Ashland, OR, USA).

TABLE 2
Primary and secondary antibodies used for cell staining

| Antibody solutions | Antibody solutions | Antibody solutions |
|---|---|---|
| against GFP tag: | against FLAG tag: | against HA tag: |
| Primary-antibody | Primary-antibody | Primary-antibody |
| rabbit pAb anti-GFP | mouse anti-FLAG | anti-HA ab |
| (PABG1) | M2 | (ab9110) |
| Secondary-antibody | Secondary-antibody | Secondary-antibody |
| Goat anti-rabbit | Goat anti-mouse | Anti-Rabbit 680 RD |
| Alexa Fluor 546 | IgG H&L Alexa 647 | |

Laser and filter parameters used in BD LSRFortessa analysis were:
1. APC-A for iRFP signal, and Antibody: Goat anti-mouse Alexa 647, Goat anti-rabbit Alexa Fluor 546
2. PE-A for tRFP signal
3. AlexaFluor488 for GFP signal, and Antibody: PGT145 Alexa 488
4. AlexaFluor700 for HA signal, and Antibody: anti-rabbit 680 RD

### Identification of Individual Signal Peptides by qPCR

qPCR was utilized to identify individual signal peptides fused into the protein of interest (POI) from cells transduced with a mixed pool SP-POI lentivirus construct. For this the gDNA of the flow-cytometer sorted cells was isolated with Nucleospin Blood Quickpure or L kits (Macherey Nagel). Enrichment of specific signal peptide-containing constructs in a specific sorted cell population (see FIG. 1) was then shown with qPCR by using signal-peptide specific primers (Table 3). The qPCR was carried out in 5 μl reaction mixture containing 75 ng gDNA, 1 μM forward and reverse primer and additional 1 mM MgC12. The conditions of the PCR were: Step 1. 95° C. 10 min, Step. 2 95° C. 15 sec., Step 3. 66° C. 5 sec, Step 4. 72° C. 10 sec, Steps 2-4 were cycled for 45 times, Step. 5 72° C. 5 min.

TABLE 3
Forward and reverse primers used for qPCR analysis of SP-GFP samples

| Name | Type | Sequence |
|---|---|---|
| fAzurocidinP_SP | Forward primer | GACCAGACTCACAGTGC SEQ |
| | | ID NO: 5 |
| fCellulaseP_SP | Forward primer | GTTCCAGAGCACTCTTTTG |
| | | SEQ ID NO: 6 |
| fFCRL1P_SP | Forward primer | GTTGCTGTTGATCTGTGC SEQ |
| | | ID NO: 7 |
| fIL4P_SP | Forward primer | CTGACCAGTCAACTCCTG |
| | | SEQ ID NO: 8 |
| fpPL_Ub_GF_SP | Forward primer | GCTGGTGGTGTCAAATCTAC |
| | | SEQ ID NO: 9 |
| fSerumAlbuminPreprotein_SP | Forward primer | GGTCACTTTCATTTCCTTGC |
| | | SEQ ID NO: 10 |
| reverse_primer_SP1-7 | Reverse primer | CGTGAAGACTCTGACTGG |
| | | SEQ ID NO: 11 |

### Identification of Signal Peptides From Large Pools by Next-Generation Sequencing

Next generation sequencing (NGS) was utilized to identify all signal peptides that have enriched into the best POI expression, showing high antibody signal flow-cytometry sorted cells population (see Fig.1). For this, first the gDNA of the flow-cytometer sorted cells was isolated with Nucleospin Blood Quickpure or L kits (Macherey Nagel). 2 μg of gDNA corresponding roughly to 360 000 copies of a diploid CHO cell genome was added to 50 μl PCR reaction mixture which contained Q5-buffer (NEB), 0.2 mM dNTPs, 0.5 mM forward and reverse primer (see Table 4), 4% DMSO, 1 U Q5 hot start polymerase (NEB) and additional 2 mM MgC12. The PCR was then carried out with the following parameters: Step 1. 98° C. 3 min, Step 2. 98° C. 30 sec, Step 3. 61° C. 15 sec, Step 4. 72° C. 15 sec, Steps 2-4 were cycled for 25 times, Step. 5. 72° C. 5 min.

The amplified, SP encoding amplicons were then size-selected with AMPure XP beads (Beckman Coulter) and finally sequenced with MiSeq sequencing (Illumina). The highest expression level conferring signal peptides were identified by comparing the read enrichment between the highest expressing 1% and the remaining 99% of the transduced, sorted cells.

TABLE 4
Forward and reverse primers used for NGS amplicon preparation from SP-GFP
samples

| Name | Type | Sequence^{1,2} |
|---|---|---|
| fIllumSSlibgen | Forward primer | AATGATACGGCGACCACCGAGATCTACACA |
| | | CACTCTTTCCCTACACGACGCTCTTCCGATC |
| | | TTAGAAGACACCGGGACCG SEQ ID NO: 12 |
| rIllumSSlibgen2 | Reverse primer | CAAGCAGAAGACGGCATACGAGATATCGC |
| | | CGTGTGACTGGAGTTCAGACGTGTGCTCTT |
| | | CCGATCTACCAGTCAGAGTCTTCACGAAG |
| | | SEQ ID NO: 13 |
| rIllumSSlibgen3 | Reverse primer | CAAGCAGAAGACGGCATACGAGATCATGA |
| | | AAGGTGACTGGAGTTCAGACGTGTGCTCTT |
| | | CCGATCTACCAGTCAGAGTCTTCACGAAG |
| | | SEQ ID NO: 14 |
| rIllumSSlibgen4 | Reverse primer | CAAGCAGAAGACGGCATACGAGATGATAG |
| | | TTGGTGACTGGAGTTCAGACGTGTGCTCTT |
| | | CCGATCTACCAGTCAGAGTCTTCACGAAG |
| | | SEQ ID NO: 15 |
| rIllumSSlibgen5 | Reverse primer | CAAGCAGAAGACGGCATACGAGATTGGTT |
| | | GCCGTGACTGGAGTTCAGACGTGTGCTCTT |
| | | CCGATCTACCAGTCAGAGTCTTCACGAAG |
| | | SEQ ID NO: 16 |
| rIllumSSlibgen6 | Reverse primer | CAAGCAGAAGACGGCATACGAGATGAAAT |
| | | GTCGTGACTGGAGTTCAGACGTGTGCTCTT |
| | | CCGATCTACCAGTCAGAGTCTTCACGAAG |
| | | SEQ ID NO: 17 |
| rIllumSSlibgen7 | Reverse primer | CAAGCAGAAGACGGCATACGAGATCTCCA |
| | | CCAGTGACTGGAGTTCAGACGTGTGCTCTT |
| | | CCGATCTACCAGTCAGAGTCTTCACGAAG |
| | | SEQ ID NO: 18 |

¹ = Illumina adapter sequence of each primer is marked by underlining.
² = The bar code index in the reverse primer's illumina adapter is shown in bold. When different sorted populations were sequenced on the same chip, different index containing reverse primers were used for the preparation of amplicons from each separate population.

### Testing of Protein Production With Transient Transfection

Elisa assay was used to show that the results of our SP screening are transferable to protein production conditions, mimicking high-level protein production in biopharmaceutical industry. Here the assay was done after identifying the highest expression level conferring signal peptides for both FVII and sfGFP. To express the aforementioned SPs containing proteins in secreted form, the protein encoding expression plasmids were transiently transfected into HEK293T or CHO cells, or other suitable mammalian expression host cells.

Depending on the target of interest, the media containing the secreted protein was harvested typically after three to five days after the transfection. Expression test samples were collected to conical tubes and centrifuged (Eppendorf) at room temperature for five minutes at 500×g, in order to pellet the cells. The cleared supernatants were placed in new tubes and the amount of the secreted sfGFP or FVII was quantified by using sandwich ELISA assay against the target of interest.

Namely, the POI or its epitope tag-binding protein was pre-coated on a 96-well ELISA plate. Harvested cleared expression media was then applied on the pre-coated plate. In addition, the separate positive and negative controls (commercial, purified POI and harvested media from mock-transfected cells, respectively) were used to verify the functionality of the assay; this was to outrule the possible false positives, and also in order to estimate the POI expression level and its functionality on the basis of its affinity against the target.

Typically the Elisa test was performed following the standard procedures recommended by manufacturers (such as Thermo-Fisher Scientific). Capture target was coated to the plate, typically overnight at 4° C. The unbound proteins were washed away with assay buffer; washing was repeated 3 times, after which the plate was briefly dried by tapping. Commercial blocking buffer (for example from Thermo-Fisher Scientific) was placed to all wells, and plate was incubated at room temperature for one hour. Blocking buffer was removed and cleared expression media and controls were added to the wells. Washing step was repeated as described previously and the wells were treated with suitable labelled antibody (Thermo-Fisher Scientific). In order to detect the protein-target complex signal, the plate was briefly air-dried by tapping against paper towel and after this 50 μl of labelled secondary detection antibody in blocking buffer was added to wells. The excess detection antibody was washed away and suitable assay buffer was added. The signals were measured by using ELISA plate reader (Thermo-Fisher Scientific).

In case needed, the HRP detection was used. In this case, instead of assay buffer, HRP conjugate and HRP substrate were added at final step, followed by the detection by using plate reader.

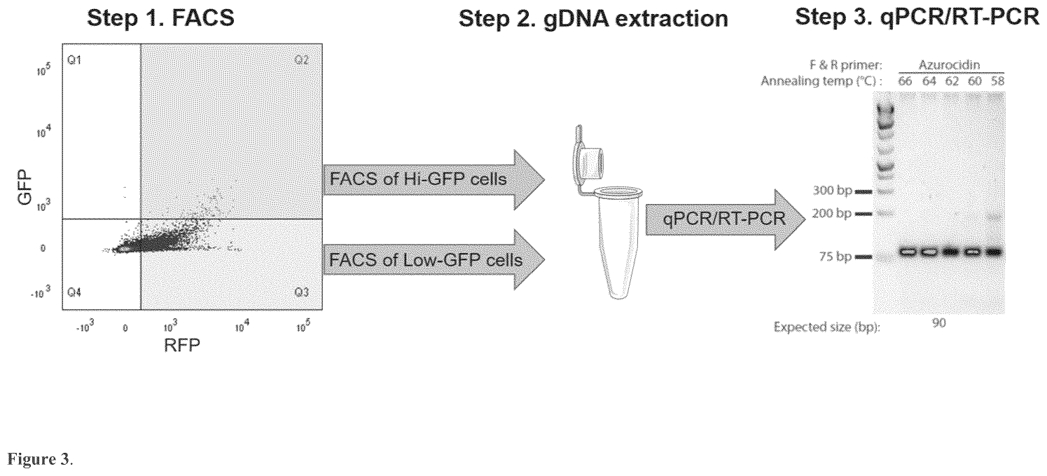

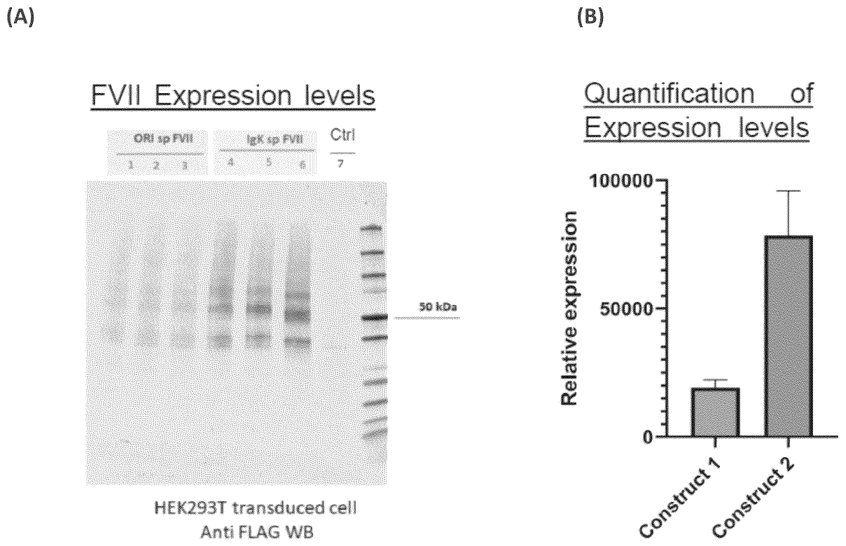

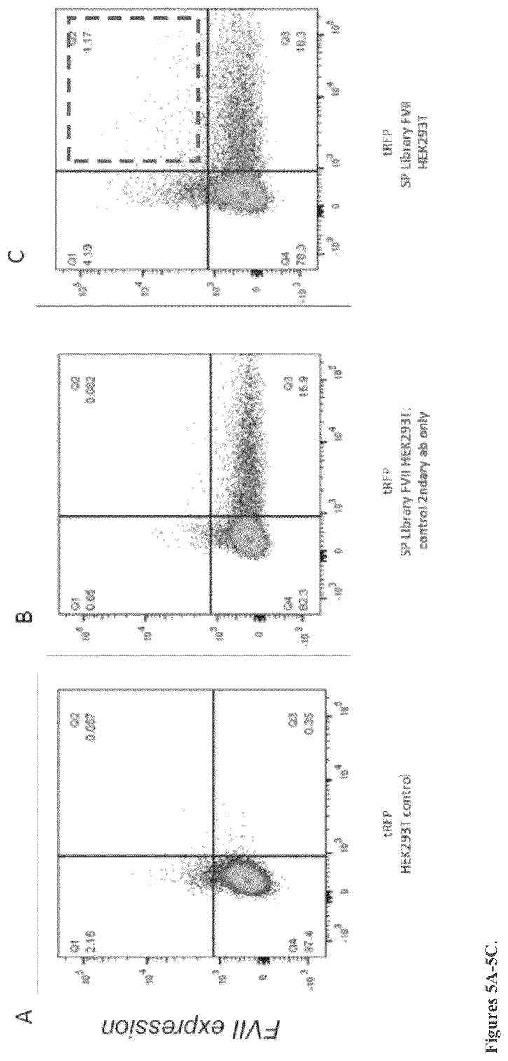

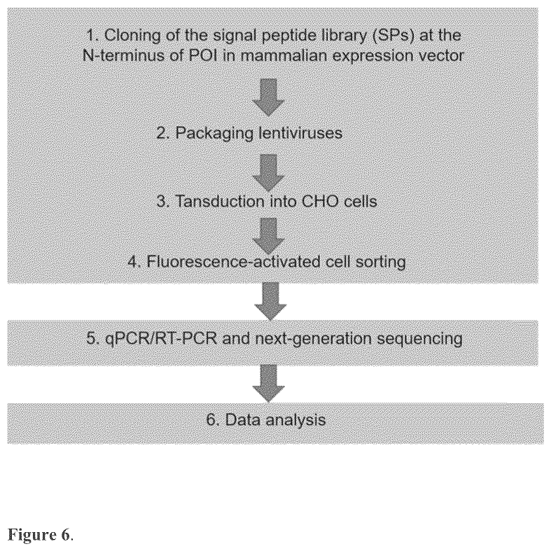

Results of the above methods are shown in FIGS. 3-5.

### REFERENCES

BASSIK, M.C., KAMPMANN, M., LEBBINK, R.J., WANG, S., HEIN, M.Y., POSER, I., WEIBEZAHN, J., HORLBECK, M.A., CHEN, S., MANN, M., HYMAN, A.A., LEPROUST, E.M., MCMANUS, M.T. and WEISSMAN, J.S., 2013. A systematic mammalian genetic interaction map reveals pathways underlying ricin susceptibility. Cell, 152(4), pp. 909-922.

BLOBEL, G. and DOBBERSTEIN, B., 1975. Transfer of proteins across membranes. I. Presence of proteolytically processed and unprocessed nascent immunoglobulin light chains on membrane-bound ribosomes of murine myeloma. The Journal of cell biology, 67(3), pp. 835-851.

COSTANTINI, L.M., BALOBAN, M., MARKWARDT, M.L., RIZZO, M., GUO, F., VERKHUSHA, V.V. and SNAPP, E.L., 2015. A palette of fluorescent proteins optimized for diverse cellular environments. Nature communications, 6, pp. 7670.

DANTUMA, N.P., LINDSTEN, K., GLAS, R., JELLNE, M. and MASUCCI, M.G., 2000. Short-lived green fluorescent proteins for quantifying ubiquitin/proteasome-dependent proteolysis in living cells. Nature biotechnology, 18(5), pp. 538-543.

GEMMER, M. and FORSTER, F., 2020. A clearer picture of the ER translocon complex. Journal of cell science, 133(3), pp. 10.1242/jcs.231340.

KOBER, L., ZEHE, C. and BODE, J., 2013. Optimized signal peptides for the development of high expressing CHO cell lines. Biotechnology and bioengineering, 110(4), pp. 1164-1173.

LOBATO-PASCUAL, A., SAETHER, P.C., FOSSUM, S., DISSEN, E. and DAWS, M.R., 2013. Mincle, the receptor for mycobacterial cord factor, forms a functional receptor complex with MCL and FcepsilonRI-gamma. European journal of immunology, 43(12), pp. 3167-3174.

MEERBREY, K.L., HU, G., KESSLER, J.D., ROARTY, K., LI, M.Z., FANG, J.E., HERSCHKOWITZ, J.I., BURROWS, A.E., CICCIA, A., SUN, T., SCHMITT, E.M., BERNARDI, R.J., FU, X., BLAND, C.S., COOPER, T.A., SCHIFF, R., ROSEN, J.M.,

WESTBROOK, T.F. and ELLEDGE, S.J., 2011. The pINDUCER lentiviral toolkit for inducible RNA interference in vitro and in vivo. Proceedings of the National Academy of Sciences of the United States of America, 108(9), pp. 3665-3670.

PENG, L., YU, X., LI, C., CAI, Y., CHEN, Y., HE, Y., YANG, J., JIN, J. and LI, H., 2016. Enhanced recombinant factor VII expression in Chinese hamster ovary cells by optimizing signal peptides and fed-batch medium. Bioengineered, 7(3), pp. 189-197.

RHEE, J.M., PIRITY, M.K., LACKAN, C.S., LONG, J.Z., KONDOH, G., TAKEDA, J. and HADJANTONAKIS, A.K., 2006. In vivo imaging and differential localization of lipid-modified GFP-variant fusions in embryonic stem cells and mice. Genesis (New York, N.Y.: 2000), 44(4), pp. 202-218.

SHCHERBAKOVA, D.M. and VERKHUSHA, V.V., 2013. Near-infrared fluorescent proteins for multicolor in vivo imaging. Nature methods, 10(8), pp. 751-754.

STRACK, R.L., STRONGIN, D.E., BHATTACHARYYA, D., TAO, W., BERMAN, A., BROXMEYER, H.E., KEENAN, R.J. and GLICK, B.S., 2008. A noncytotoxic DsRed variant for whole-cell labeling. Nature methods, 5(11), pp. 955-957.

TAN, N.S., HO, B. and DING, J.L., 2002. Engineering a novel secretion signal for cross-host recombinant protein expression. Protein engineering, 15(4), pp. 337-345.

TISCORNIA, G., SINGER, O. and VERMA, I.M., 2006. Production and purification of lentiviral vectors. Nature protocols, 1(1), pp. 241-245.

VON HEIJNE, G., 1985. Signal sequences. The limits of variation. Journal of Molecular Biology, 184(1), pp. 99-105.

1. A method for screening a signal peptide for efficient expression and secretion of a heterologous polypeptide in mammalian cells, the method comprising the steps of:
a) providing a pool of viral expression vectors encoding a polypeptide of interest with various candidate signal peptides, wherein each viral expression vector of said pool comprises at least a polynucleotide encoding a fusion protein, said polynucleotide comprising: i) a promoter, ii) a sequence encoding a signal peptide, iii) a sequence encoding the polypeptide of interest, iv) optionally a sequence encoding an epitope tag, and v) a sequence encoding a C-terminal signal peptide for glycosylphosphatidylinositol, GPI, attachment or a sequence encoding a transmembrane domain;
b) transforming host cells with said pool of viral vectors so that each host cell is transformed on average by only one viral vector from said pool;
c) expressing said fusion protein in said host cells in order to produce fusion proteins which are GPI-anchored to the host cell surface or alternatively which are anchored to the host cell surface by a transmembrane domain of said fusion protein;
d) contacting said host cells with a first binding reagent specifically binding to said polypeptide of interest or, alternatively, if said epitope tag is present in the fusion protein with a first binding reagent specifically binding to said epitope tag, wherein said binding reagent is optionally labelled with a fluorescent label or other means of detection;
e) optionally contacting said host cells with a second binding reagent labelled with a fluorescent label if said first binding reagent is not a labelled binding reagent, wherein said second binding reagent is specifically binding to said first binding reagent;
f) dividing the transformed host cells into at least two groups based on the fluorescence characteristics of each host cell;
g) performing next generation sequencing to the group of host cells showing the most efficient fusion protein expression in order to identify an optimal signal peptide for the polypeptide of interest in the host cell.

2. The method according to claim 1, wherein said pool of viral expression vectors encoding a polypeptide of interest is prepared by:
synthesizing a library of oligonucleotides encoding various signal peptides and complementary sequences thereof, wherein said oligonucleotides form restriction site overhangs at both free ends of the sequence when bound to a complementary oligonucleotide in the library;
annealing the oligonucleotides in the library with the complementary oligonucleotides present in the library to produce double-stranded DNA fragments with said restriction site overhangs; and
ligating said double-stranded DNA fragments to a viral vector having a suitable cloning site to combine the DNA fragment encoding a signal peptide with a polynucleotide encoding a protein of interest and thus producing a pool of viral expression vectors encoding a polypeptide of interest with various candidate signal peptides.

3. The method according to claim 2, wherein the signal peptides are selected from the known signal peptides of eukaryotic proteins, modifications thereof and/or artificial sequences.

4. The method according to claim 1 comprising a further step of:
h) cloning a polynucleotide encoding the combination of said optimal signal peptide detected in step g) and the polypeptide of interest to a second expression vector, transforming a host cell with said second vector and producing said polypeptide in said host cell.

5. The method according to claim 1, wherein said host cell is a cell selected from the group consisting of a CHO cell, HeLa cell, HEK293 cell, BHK cell, COS7 cell, COPS cell, A549 cell, NIH3T3 cell, MDCK cell and WI38 cell.

6. The method according to claim 1, wherein said viral vector is a lentiviral vector.

7. The method according to claim 1, wherein said promoter is an inducible promoter.

8. The method according to claim 1, wherein said epitope tag is selected from the group consisting of Histidine tag (His-tag), myc-tag, FLAG-tag, small ubiquitin-like modifier tag (SUMO-tag), a heavy chain of protein C tag (HPC-tag), a calmodulin binding peptide tag (CBP-tag), and a hemagglutinin-tag (HA-tag) or other epitope tags or other labeling groups.

9. The method according to claim 1, wherein said polypeptide of interest is a therapeutic or diagnostic protein.

10. The method according to claim 1, wherein step f) is performed using fluorescence-activated cell sorting, FACS.

11. The method according to claim 1, wherein each viral expression vector of said pool further comprises a polynucleotide encoding a transduction marker protein.

12. A viral expression vector comprising at least a polynucleotide encoding a fusion protein, said polynucleotide comprising: i) a promoter, ii) a sequence encoding a signal peptide, iii) a sequence encoding a polypeptide of interest, iv) optionally a sequence encoding an epitope tag, and v) a sequence encoding a C-terminal signal peptide for glycosylphosphatidylinositol, GPI, attachment or a sequence encoding a transmembrane domain.

13. The vector according to claim 12, wherein said inducible promoter is a tetracycline controlled promoter or other suitable promoter sequence including constitutive promoters.

14. The vector according to claim 12, wherein said epitope tag is selected from the group consisting of Histidine tag (His-tag), myc-tag, FLAG-tag, small ubiquitin-like modifier tag (SUMO-tag), a heavy chain of protein C tag (HPC-tag), a calmodulin binding peptide tag (CBP-tag), and a hemagglutinin-tag (HA-tag) or other epitope tags or other labeling groups

15. The vector according to claim 12, wherein said polypeptide of interest is a therapeutic protein-such-as-a-therapeutic-antibody.

16. The vector according to claim 12, wherein said vector is a lentiviral vector.

17. The vector according to claim 12, further comprising a sequence encoding a transduction marker protein.

18. A host cell comprising a viral vector according to claim 12.

19. The host cell according to claim 18, the host cell presenting a fusion protein on its cell membrane surface, wherein said fusion protein is attached to the cell membrane by GPI attachment or via a transmembrane domain.

20. The host cell according to claim 18, wherein said host cell is selected from the group consisting of a CHO cell, HeLa cell, HEK293 cell, BHK cell, COS7 cell, COP5 cell, A549 cell, NIH3T3 cell, MDCK cell and WI38 cell.

21. A DNA library comprising multiple viral vectors according to claim 12, wherein the vectors encode candidate signal peptides for a polypeptide of interest.


SEQUENCE LISTING (18 sequences)

SEQ ID NO: 1 - protein, 20 aa, synthetic construct
  REGION 1..20: note = Signal peptide used with FVII expression
METDTLLLWVLLLWVPGSTG

SEQ ID NO: 2 - DNA, 60 bp, synthetic construct
  misc_feature 1..60: note = Codon optimized DNA sequence
  source 1..60: mol_type = other DNA
atggagaccgacaccctgctgctgtgggtgctgctgctgtgggtgcccggctccaccggc

SEQ ID NO: 3 - protein, 20 aa, synthetic construct
  REGION 1..20: note = Signal peptide used with FVII expression
MVSQALRLLCLLLGLQGCLA

SEQ ID NO: 4 - DNA, 60 bp, synthetic construct
  misc_feature 1..60: note = Codon-optimized DNA sequence
  source 1..60: mol_type = other DNA
atggtgtcccaggccctgaggctgctgtgcctgctgctgggcctgcagggctgcctggcc

SEQ ID NO: 5 - DNA, 17 bp, synthetic construct
  misc_feature 1..17: note = Oligonucleotide primer
  source 1..17: mol_type = other DNA
gaccagactcacagtgc

SEQ ID NO: 6 - DNA, 19 bp, synthetic construct
  misc_feature 1..19: note = Oligonucleotide primer
  source 1..19: mol_type = other DNA
gttccagagcactcttttg

SEQ ID NO: 7 - DNA, 18 bp, synthetic construct
  misc_feature 1..18: note = Oligonucleotide primer
  source 1..18: mol_type = other DNA
gttgctgttgatctgtgc

SEQ ID NO: 8 - DNA, 18 bp, synthetic construct
  misc_feature 1..18: note = Oligonucleotide primer
  source 1..18: mol_type = other DNA
ctgaccagtcaactcctg

SEQ ID NO: 9 - DNA, 20 bp, synthetic construct
  misc_feature 1..20: note = Oligonucleotide primer
  source 1..20: mol_type = other DNA
gctggtggtgtcaaatctac

SEQ ID NO: 10 - DNA, 20 bp, synthetic construct
  misc_feature 1..20: note = Oligonucleotide primer
  source 1..20: mol_type = other DNA
ggtcactttcatttccttgc

SEQ ID NO: 11 - DNA, 18 bp, synthetic construct
  misc_feature 1..18: note = Oligonucleotide primer
  source 1..18: mol_type = other DNA
cgtgaagactctgactgg

SEQ ID NO: 12 - DNA, 80 bp, synthetic construct
  misc_feature 1..80: note = Oligonucleotide primer
  source 1..80: mol_type = other DNA
aatgatacggcgaccaccgagatctacacacactctttccctacacgacgctcttccgatcttagaagacaccgggaccg

SEQ ID NO: 13 - DNA, 88 bp, synthetic construct
  misc_feature 1..88: note = Oligonucleotide primer
  source 1..88: mol_type = other DNA
caagcagaagacggcatacgagatatcgccgtgtgactggagttcagacgtgtgctcttccgatctaccagtcagagtcttcacgaag

SEQ ID NO: 14 - DNA, 88 bp, synthetic construct
  misc_feature 1..88: note = Oligonucleotide primer
  source 1..88: mol_type = other DNA
caagcagaagacggcatacgagatcatgaaaggtgactggagttcagacgtgtgctcttccgatctaccagtcagagtcttcacgaag

SEQ ID NO: 15 - DNA, 88 bp, synthetic construct
  misc_feature 1..88: note = Oligonucleotide primer
  source 1..88: mol_type = other DNA
caagcagaagacggcatacgagatgatagttggtgactggagttcagacgtgtgctcttccgatctaccagtcagagtcttcacgaag

SEQ ID NO: 16 - DNA, 88 bp, synthetic construct
  misc_feature 1..88: note = Oligonucleotide primer
  source 1..88: mol_type = other DNA
caagcagaagacggcatacgagattggttgccgtgactggagttcagacgtgtgctcttccgatctaccagtcagagtcttcacgaag

SEQ ID NO: 17 - DNA, 88 bp, synthetic construct
  misc_feature 1..88: note = Oligonucleotide primer
  source 1..88: mol_type = other DNA
caagcagaagacggcatacgagatgaaatgtcgtgactggagttcagacgtgtgctcttccgatctaccagtcagagtcttcacgaag

SEQ ID NO: 18 - DNA, 88 bp, synthetic construct
  misc_feature 1..88: note = Oligonucleotide primer
  source 1..88: mol_type = other DNA
caagcagaagacggcatacgagatctccaccagtgactggagttcagacgtgtgctcttccgatctaccagtcagagtcttcacgaag